(19) United States
(12) Patent Application Publication
(10) Pub. No.: US 2025/0339595 A1
(43) Pub. Date: Nov. 6, 2025

(54) CONDUCTIVITY SENSOR

(71) Applicant: MOZARC MEDICAL US LLC, Minneapolis, MN (US)

(72) Inventors: Giuliano Giganti, Mirandola (IT); Niccolo Chelli, Mirandola (IT); Tommaso Montanari, Mirandola (IT); Francesco Tarantini, Mirandola (IT)

(21) Appl. No.: 19/198,933

(22) Filed: May 5, 2025

Related U.S. Application Data
(60) Provisional application No. 63/643,117, filed on May 6, 2024.

(51) Int. Cl. A61M 1/28; G01N 27/07; G01N 27/08; H01B 1/02

(52) U.S. Cl. CPC A61M 1/28; G01N 27/07; G01N 27/08; H01B 1/02; A61M 2205/3327; A61M 2205/50

(57) ABSTRACT

A conductivity sensor includes a housing including a fluid inlet, a fluid outlet; and a chamber having an inner wall. The chamber is located between the fluid inlet and the fluid outlet. A conductivity sensor includes a first electrode. At least a portion of the first electrode is located in the chamber. The first electrode has a first longitudinal axis. The first electrode does not form any portion of the inner wall of the chamber. A conductivity sensor includes a second electrode. The second electrode forms at least a portion of the inner wall of the chamber. The second electrode has a second longitudinal axis. The first longitudinal axis is coaxial with the second longitudinal axis. A flow path of a fluid through the conductivity sensor is from the fluid inlet through the chamber to the fluid outlet.

### CROSS REFERENCE

This application claims the benefit of and priority to U.S. Provisional Patent Application No. 63/643,117 filed May 6, 2024, the entire content of each of which is incorporated by reference herein.

### FIELD

This disclosure relates generally to a conductivity sensor. More particularly, this disclosure relates to a conductivity sensor configured for use in a dialysis system.

### BACKGROUND

Dialysis systems can be used to treat patients with kidney disorders. There are a number of dialysis systems in use in the health care industry. Dialysis fluids that are specifically controlled for the dialysis systems are used in these dialysis systems for treatment of the patients.

### SUMMARY

In some embodiments, a conductivity sensor includes a housing. In some embodiments, the housing includes a fluid inlet; a fluid outlet; and a chamber having an inner wall. In some embodiments, the chamber is located between the fluid inlet and the fluid outlet. In some embodiments, the conductivity sensor includes a first electrode. In some embodiments, at least a portion of the first electrode is located in the chamber. In some embodiments, the first electrode has a first longitudinal axis. In some embodiments, the first electrode does not form any portion of the inner wall of the chamber. In some embodiments, the conductivity sensor includes a second electrode. In some embodiments, the second electrode forms at least a portion of the inner wall of the chamber. In some embodiments, the second electrode has a second longitudinal axis. In some embodiments, the first longitudinal axis is coaxial with the second longitudinal axis. In some embodiments, a flow path of a fluid through the conductivity sensor is from the fluid inlet through the chamber to the fluid outlet.

In some embodiments, the fluid inlet has a third longitudinal axis. In some embodiments, the fluid outlet has a fourth longitudinal axis. In some embodiments, the third longitudinal axis is parallel to the fourth longitudinal axis. In some embodiments, wherein the third longitudinal axis is offset from the fourth longitudinal axis.

In some embodiments, the third longitudinal axis is coaxial with the first longitudinal axis.

In some embodiments, an outer surface of the second electrode includes a plurality of protrusions. In some embodiments, an inner surface of the housing includes a plurality of grooves. In some embodiments, the plurality of grooves is configured to receive the plurality of protrusions when the second electrode is installed in the housing.

In some embodiments, at least one of the first electrode or the second electrode includes titanium.

In some embodiments, the housing includes an output connector configured to be connected to a processor.

In some embodiments, the processor is configured to be secured to the housing.

In some embodiments, a first end of the first electrode is configured to be disposed adjacent to a first end of the second electrode. In some embodiments, the first end of the second electrode is disposed downstream of a second end of the second electrode.

In some embodiments, the first electrode is a first cylindrically shaped electrode having a first diameter, the second electrode is a second cylindrically shaped electrode having a second diameter, and the second diameter is greater than the first diameter.

In some embodiments, the conductivity sensor is configured to bring the fluid flowing through the chamber into contact with the first electrode and the second electrode.

In some embodiments, a dialysis system includes a water purification system configured to purify a fluid for use in dialysis. In some embodiments, the water purification system includes a conductivity sensor configured to sense a conductivity of the fluid for use in dialysis. In some embodiments, the conductivity sensor includes a housing including a fluid inlet; a fluid outlet; and a chamber having an inner wall. In some embodiments, the chamber is located between the fluid inlet and the fluid outlet. In some embodiments, the conductivity sensor includes a first electrode. In some embodiments, at least a portion of the first electrode is located in the chamber. In some embodiments, the first electrode does not form any portion of the inner wall of the chamber. In some embodiments, the conductivity sensor includes a second electrode. In some embodiments, the second electrode forms at least a portion of the inner wall of the chamber. In some embodiments, a flow path of the fluid for use in dialysis through the conductivity sensor is from the fluid inlet through the chamber to the fluid outlet.

In some embodiments, the first electrode has a first longitudinal axis. In some embodiments, the second electrode has a second longitudinal axis. In some embodiments, the first longitudinal axis is coaxial with the second longitudinal axis. In some embodiments, the fluid outlet has a third longitudinal axis. In some embodiments, the third longitudinal axis is coaxial with the first longitudinal axis.

In some embodiments, the fluid inlet has a fourth longitudinal axis. In some embodiments, wherein the third longitudinal axis is offset from the fourth longitudinal axis.

In some embodiments, an outer surface of the second electrode includes a plurality of protrusions. In some embodiments, an inner surface of the housing includes a plurality of grooves. In some embodiments, the plurality of grooves is configured to receive the plurality of protrusions when the second electrode is installed in the housing.

In some embodiments, at least one of the first electrode or the second electrode includes titanium.

In some embodiments, the housing includes an output connector configured to be connected to a processor.

In some embodiments, the processor is configured to be secured to the housing.

In some embodiments, a first end of the first electrode is configured to be disposed adjacent a first end of the second electrode. In some embodiments, the first end of the second electrode is disposed downstream of a second end of the second electrode.

In some embodiments, a second conductivity sensor is configured to sense a conductivity of the fluid for use in dialysis at a different location in the water purification system than the conductivity sensor.

In some embodiments, the conductivity sensor is configured to determine a first error condition in which a concentration of ions in the fluid for use in dialysis is greater than an ion concentration threshold.

### BRIEF DESCRIPTION OF THE DRAWINGS

References are made to the accompanying drawings that form a part of this disclosure and that illustrate embodiments in which the systems and methods described in this Specification can be practiced.

FIG. 1 is a schematic diagram of a dialysis system, according to some embodiments.

FIG. 2 is a schematic diagram of the water filtration system of FIG. 1, according to some embodiments.

FIG. 3 is a schematic diagram of the pre-filtration system for the water filtration

system of FIG. 1, according to some embodiments.

FIG. 4 is a schematic diagram of the treatment system for the water filtration system of FIG. 1, according to some embodiments.

FIG. 5 is a schematic diagram of the distribution system for the water filtration system of FIG. 1, according to some embodiments.

FIG. 6 is a flowchart of a method for controlling a dialysis system, according to some embodiments.

FIG. 7 shows a perspective view of a conductivity sensor, according to some embodiments.

FIG. 8 shows a sectional view of the conductivity sensor of FIG. 7, according to some embodiments.

Like reference numbers represent the same or similar parts throughout.

### DETAILED DESCRIPTION

Dialysis systems such as, but not limited to, hemodialysis, hemofiltration, hemodiafiltration, and peritoneal dialysis, can utilize a high volume water source that is purified to reach purity levels needed for the dialysis treatments. The water filtration systems disclosed are designed to reduce risk of contaminants and to ensure an appropriate composition of the purified water.

Embodiments of this disclosure are directed to improved systems and methods for ensuring that the water used to generate the dialysate for the dialysis process meets appropriate purity goals. In some embodiments, these can be determined based on inferences using conductivity sensors, pressure sensors, flowrate sensors, combinations thereof, or the like. In some embodiments, total organic carbon (TOC) sensors can be used. In response to determining that one or more of the sensed values are outside of a threshold, a controller for the water filtration system can change a state of a valve (e.g., open the valve) to drain the water from the system. In some embodiments, the water can be drained at a location that is downstream of the identified problem and upstream of additional filtration steps. In some embodiments, the water can be drained at a location upstream of the identified problem.

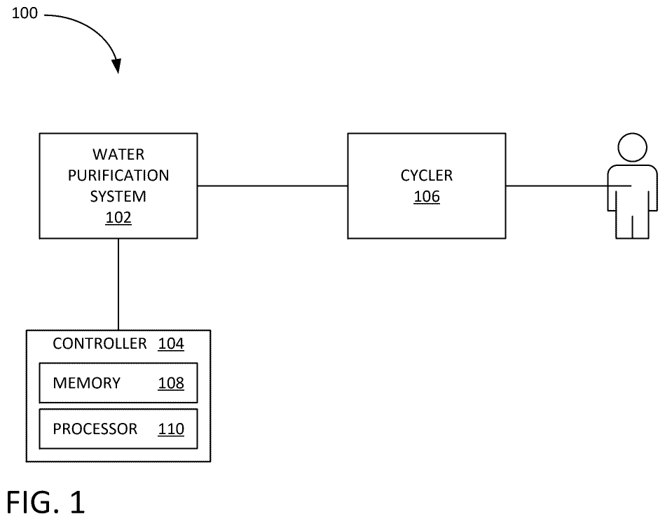

FIG. 1 is a schematic diagram of a dialysis system 100, according to some embodiments. In some embodiments, the dialysis system 100 can be representative of a peritoneal dialysis system including point of use dialysis fluid production. Peritoneal dialysis systems are one example of a dialysis system. It is to be appreciated that the systems and methods described in this disclosure can be applied to other dialysis systems such as, but not limited to, hemodialysis, hemofiltration, hemodiafiltration, or the like.

The illustrated embodiment includes a water purification system 102. A controller 104 is configured to be in electronic communication with the water purification system 102 to send and receive communications relating to sensed parameters, control of valves, or the like. The water purification system 102 can be fluidly connected to a cycler 106. The cycler 106 can be fluidly connected with a patient to perform the dialysis treatments. The cycler 106 can be configured to inject the dialysis fluid into the patient and drain the dialysis fluid when the treatment is complete. The cycler 106 can be in electronic communication with the controller 104 to accomplish the necessary treatments for the patient. In some embodiments, another device in the water purification system 102 may prepare the fresh dialysis fluid using purified water output from the water purification system 102. For example, the water purification system 102 may include a preparator for mixing the fresh dialysis fluid using purified water. The preparator can be in electronic communication with the controller 104 and the cycler 106 to accomplish the necessary treatments for the patient. In some embodiments, the preparator can be in electronic communication with the cycler 106 to accomplish the necessary treatments for the patient. It is to be appreciated that the cycler 106 can include one or more additional features such as, but not limited to, a user interface configured to receive user inputs, display outputs for the user, or any combination thereof.

The controller 104 can be in wired or wireless communication with the water purification system 102. The controller 104 can include a memory 108 and at least one processor 110. It is to be appreciated that the controller 104 can include one or more additional features such as, but not limited to, a display with a user interface configured to receive user inputs, display outputs for the user, or any combination thereof. In some embodiments, a separate user input can also be included so the user can interact with the dialysis system 100.

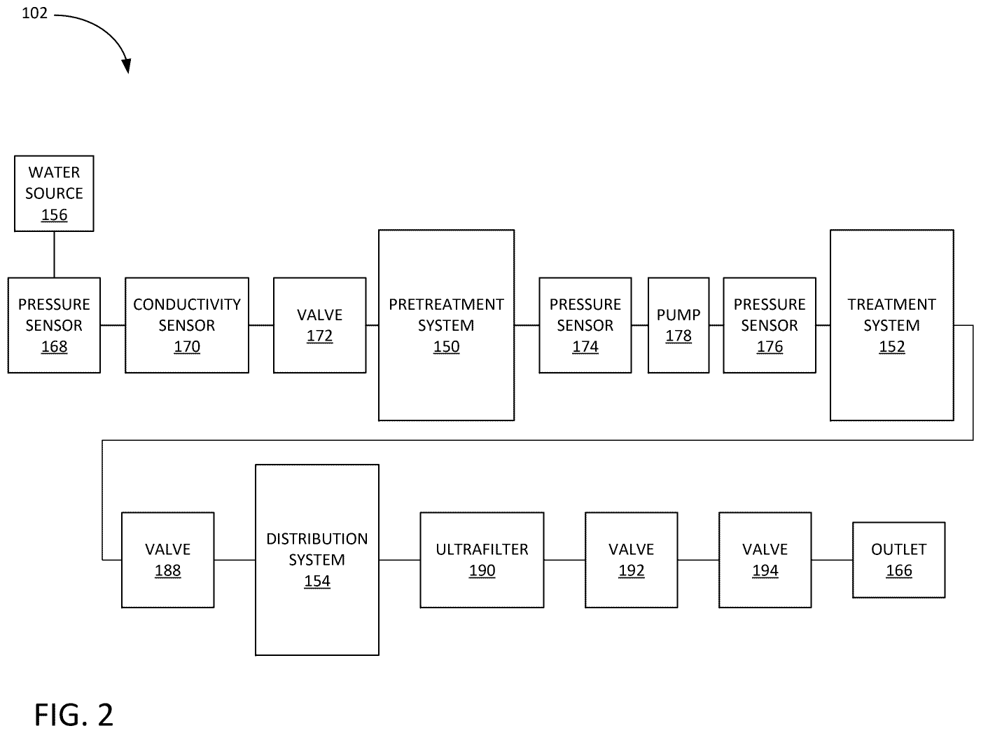

FIG. 2 is a schematic diagram of the water purification system 102, according to some embodiments. In some embodiments, the water purification system 102 can be broken down into subsystems including a pretreatment system 150, a treatment system 152, and a distribution system 154. In some embodiments, the water purification system 102 can be contained within an apparatus that is fluidly connected to the cycler 106.

The water purification system 102 is fluidly connected to a water source 156. For example, the water source 156 can be a water tap or the like. The fluid received at the water purification system 102 from the water source 156 can be treated using the pretreatment system 150, the treatment system 152, and the distribution system 154.

In some embodiments, the distribution system 154 includes an outlet 166 configured to be fluidly connected to the cycler 106.

In some embodiments, one or more additional components can be included in the water purification system 102. For example, the water purification system 102 can include a pressure sensor 168, a conductivity sensor 170, and a valve 172 fluidly disposed between the water source 156 and the pretreatment system 150. In some embodiments, a pressure sensor 174 and a pressure sensor 176 can be disposed fluidly between the pretreatment system 150 and the treatment system 152. In some embodiments, a pump 178 can be disposed fluidly between the pressure sensor 174 and the pressure sensor 176. In some embodiments, a valve 188 can be disposed fluidly between the treatment system 152 and the distribution system 154.

In some embodiments, an ultrafilter 190, a valve 192, and a valve 194 can be disposed between the distribution system 154 and the cycler 106 (FIG. 1).

In some embodiments, the valve 172, the valve 188, the valve 192, and the valve 194 can be electronically controlled valves in electronic communication with the controller 104 of the water purification system 102. In some embodiments, the valve 172, the valve 188, the valve 192, and the valve 194 can be selectively activated to drain the water from the water purification system 102. As such, although not shown in the figure, the valve 172, the valve 188, the valve 192, and the valve 194 can also be fluidly connected with a drain of the water purification system 102.

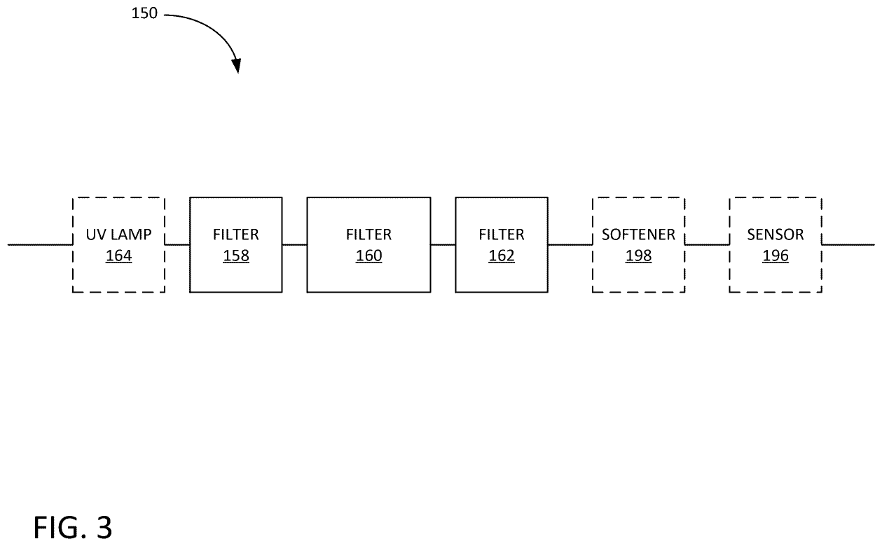

In some embodiments, the controller 104 can be configured to receive inputs from the pressure sensor 168, the conductivity sensor 170, the pressure sensor 174, and the pressure sensor 176 (in addition to other sensors shown and described in additional detail in FIGS. 3-5 below) to selectively drain water from the water purification system 102 via opening of one or more of the valve 172, the valve 188, the valve 192, and the valve 194. For example, in some embodiments, if a condition is detected that indicates that one or more parameters of the water are not being met by the pretreatment system 150, the treatment system 152, or the distribution system 154, the controller 104 can selectively open one of the valves to ensure that water not meeting purity requirements is not output to the cycler 106. In some embodiments, the controller 104 can be configured to receive inputs from the conductivity sensor 170 and the sensor 196 (shown in FIG. 3) to determine a water quality of the fluid, and the controller 104 may be configured to receive inputs from the pressure sensor 168, the conductivity sensor 170, the pressure sensor 174, and the pressure sensor 176 (in addition to other sensors shown and described in additional detail in FIGS. 3-5 below) to selectively drain water from the water purification system 102 via opening of one or more of the valve 172, the valve 188, the valve 192, and the valve 194.

In some embodiments, by being able to open a variety of valves for this purpose, it is possible to prevent the water from unnecessarily going through components of the water purification system 102, which can prolong a lifetime of those components when an upstream failure occurs. Additionally, in this manner, it is possible to maintain a required purity of the water during the course of filtration. In some embodiments, this can provide a real-time understanding of whether the water meets the purity requirements for the dialysis system 100.

In some embodiments, one or more additional conductivity sensors can be located within the pretreatment system 150, the treatment system 152, the distribution system 154, or combinations thereof.

FIG. 3 is a schematic diagram of the pretreatment system 150 for the water purification system 102, according to some embodiments. In some embodiments, the pretreatment system 150 can be configured to reduce bacteria and sediment, filter coarse particles, reduce hardness, and remove heavy metals from the water received via the water source 156.

In some embodiments, the pretreatment system 150 includes a first filter 158, a second filter 160, and a third filter 162 connected in series. In some embodiments, the pretreatment system 150 can additionally include an ultraviolet (UV) lamp 164 to disinfect the water stream. In some embodiments, the lamp 164 can be included instead of the first filter 158.

In some embodiments, the second filter 160 and the third filter 162 can be the same filters. That is, in some embodiments, the third filter 162 can be redundant to the second filter 160.

In some embodiments, the second filter 160, the third filter 162, or both the second filter 160 and the third filter 162 can be an activated carbon filter. In some embodiments, the second filter 160 and the third filter 162 can reduce a concentration of chlorine and chloramine in the water. In some embodiments, the third filter 162 can serve to act in case of a failure by the second filter 160.

In some embodiments, the third filter 162 can be used to remove endotoxins from the water. In some embodiments, the lamp 164 can be used to kill bacteria in the water. In some embodiments, the lamp 164 can be located upstream of first filter 158. In other embodiments, the lamp 164 can be located downstream of third filter 162. In yet other embodiments, the lamp 164 can be located between first filter 158 and second filter 160, between second filter 160 and third filter 162, between third filter 162 and sensor 196, or other locations.

In some embodiments, the first filter 158, the second filter 160, and the third filter 162 can be configured to collectively remove particles from the water. In some embodiments, the particles being removed can include clay, silt, silicon, combinations thereof, or the like.

In some embodiments, the first filter 158, the second filter 160, and the third filter 162 can be configured to collectively remove contaminants from the water. In some embodiments, the contaminants can include, but is not limited to, chlorine and compositions including chlorine from the water. In some embodiments, the first filter 158, the second filter 160, and the third filter 162 can be configured to collectively absorb toxic substances such as, but not limited to, pesticides. In some embodiments, the first filter 158, the second filter 160, and the third filter 162 can be configured to collectively remove hypochlorite, chloramine, and chlorine from the water. It is to be appreciated that the number of filters, functionality, and position in the pretreatment system 150 can vary and can be dependent on the application.

In some embodiments, the pretreatment system 150 can additionally include a sensor 196 disposed downstream of the third filter 162. In some embodiments, the sensor 196 can be used to assess performance of the second filter 160 and the third filter 162. In some embodiments, the sensor 196 can provide an estimate of levels of organic contamination in the water exiting the water purification system 102. In some embodiments, the controller 104 (FIG. 2) can be configured to change a state of the valve 172 (FIG. 2) if the reading from the sensor 196 is greater than a threshold value. In some embodiments, the sensor 196 can be in-line with the other filters in the pretreatment system 150. In other embodiments, one or more of the other components in the pretreatment system 150 may include the sensor 196 to measure a concentration of contaminants including organic carbon in the fluid. In some embodiments, being greater than the threshold value can be an indication that contaminant removal is not reaching required levels. For example, in some embodiments, the contaminants can include, but is not limited to, chlorine, chloramine, or other contaminants including organic carbon and being greater than the threshold value can be an indication that the removal of contaminants containing organic carbon is not reaching required levels. It is to be appreciated that the sensor 196 may not directly identify whether chlorine, chloramine, or organic carbon are passing through the pretreatment system 150 but can give an indication that the water purification system 102, or one or more components of the water purification system 102 is not working effectively. For example, in some embodiments, the sensor 196 can give an indication that one or both of the second filter 160 and the third filter 162 are not working effectively.

In some embodiments, the pretreatment system 150 can additionally include a softener 198. In some embodiments, the softener 198 can be located downstream of third filter 162. In other embodiments, the softener 198 can be located between two of the first filter 158, second filter 160, third filter 162, and the sensor 196. In yet other embodiments, the softener 198 can be located upstream of first filter 158.

In some embodiments, causing a drain of the system can also include providing an output from the controller 104 (FIG. 2) to generate an alert to indicate that the water purification system 102 is not working properly and may need to be serviced.

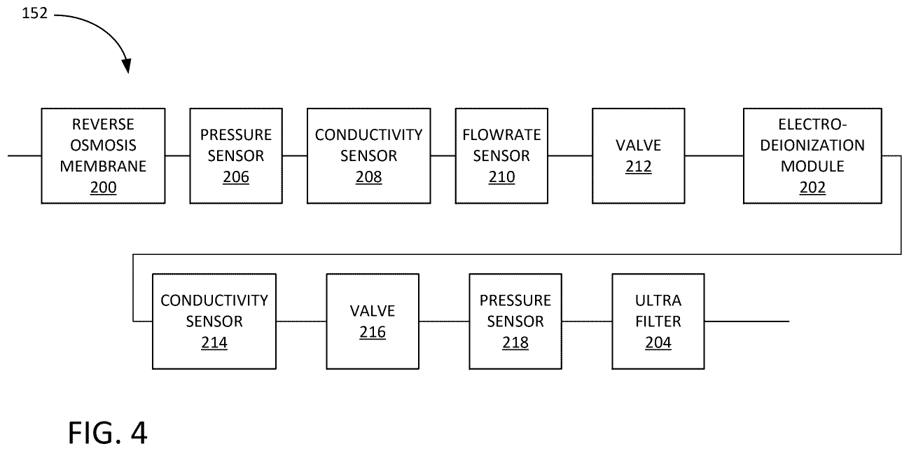

FIG. 4 is a schematic diagram of the treatment system 152 for the water purification system 102, according to some embodiments. In some embodiments, the treatment system 152 can include a reverse osmosis membrane 200, an electro-deionization module 202, and an ultrafilter 204.

In some embodiments, the treatment system 152 includes one or more additional components. In some embodiments, the treatment system 152 can include a pressure sensor 206, a conductivity sensor 208, a flowrate sensor 210, and a valve 212 fluidly disposed downstream of the reverse osmosis membrane 200 and upstream of the electro-deionization module 202.

In some embodiments, a conductivity sensor 214, a valve 216, and a pressure sensor 218 can be disposed fluidly downstream of the electro-deionization module 202 and fluidly upstream of the ultrafilter 204.

In some embodiments, the valve 188 (FIG. 2) can be disposed fluidly downstream of the ultrafilter 204.

In some embodiments, the controller 104 can be configured to monitor at least one of the pressure sensor 206, conductivity sensor 208, and flowrate sensor 210. In some embodiments, monitoring these components of the treatment system 152 can provide an understanding of whether the reverse osmosis membrane 200 is functioning properly. In some embodiments, if, for example, aluminum is passing through the reverse osmosis membrane 200, a conductivity measured by the conductivity sensor 208 would be higher than if the reverse osmosis membrane 200 is functioning properly. In such embodiments, an efficiency of the reverse osmosis membrane 200 may be reduced compared to a properly functioning reverse osmosis membrane 200. In some embodiments, the conductivity sensor 208 can accordingly be used to infer whether the treatment system 152 is properly removing metals such as, but not limited to, aluminum. In some embodiments, if the conductivity as measured by the conductivity sensor 208 is higher than a threshold conductivity, the controller 104 (FIG. 1) can be configured to change a state of the valve 212 to drain the water from the water purification system 102 and prevent water from continuing through the treatment system 152. In some embodiments, the controller 104 (FIG. 1) can be configured to change a state of the valve 172 (FIG. 2) instead of, or in addition to, the valve 212 to prevent water from depleting the filters in the pretreatment system 150 (FIG. 2) if a suspected problem is identified with the reverse osmosis membrane 200. In some embodiments, the controller 104 (FIG. 1) may make the decision based on a combination of the readings from the pressure sensor 206, the conductivity sensor 208 and the flowrate sensor 210.

In some embodiments, the controller 104 (FIG. 1) can be configured to monitor the conductivity sensor 214. In some embodiments, like the reverse osmosis membrane 200, the electro-deionization module 202 is configured to remove metals such as, but not limited to, aluminum from the water. In some embodiments, if the conductivity as measured at conductivity sensor 214 is higher than a threshold value, then it can be inferred that more metal content is passing through the electro-deionization module 202 than desired. As a result, the controller 104 (FIG. 1) can be configured to open the valve 212 and drain the water from the water purification system 102. In some embodiments, the controller 104 (FIG. 1) can be configured to change a state of the valve 172 (FIG. 2) instead of, or in addition to, the valve 212 to prevent water from depleting the filters in the pretreatment system 150 if a suspected problem is identified with the electro-deionization module 202.

In some embodiments, the reverse osmosis membrane 200 and the electro-deionization module 202 can generally be configured to control a concentration of ions in the water being filtered. In some embodiments, the reverse osmosis membrane 200 and the electro-deionization module 202 can be configured to control a concentration of nitrates in the water being filtered. In some embodiments, if the concentration of ions in the water is higher than desired, the conductivity will also be higher than expected. In some embodiments, if the concentration of nitrates in the water is higher than desired, the conductivity will also be higher than expected. As a result, readings from the conductivity sensor 208 can be used to infer whether the reverse osmosis membrane 200 is properly functioning and removing nitrates as expected. If the conductivity is higher than a threshold value, the controller 104 can be configured to change a state of the valve 212 to drain the water from the water purification system 102 and prevent water from continuing through the treatment system 152. In some embodiments, the controller 104 (FIG. 1) can be configured to change a state of the valve 172 (FIG. 2) instead of, or in addition to, the valve 212 to prevent water from depleting the filters in the pretreatment system 150 (FIG. 2) if a suspected problem is identified with the reverse osmosis membrane 200.

In some embodiments, the controller 104 (FIG. 1) can be configured to monitor the conductivity sensor 214. In some embodiments, like the reverse osmosis membrane 200, the electro-deionization module 202 is configured to remove nitrates from the water being filtered. In some embodiments, if the conductivity as measured at conductivity sensor 214 is higher than a threshold value, then it can be inferred that more nitrates are passing through the electro-deionization module 202 than desired. As a result, the controller 104 (FIG. 1) can be configured to open the valve 212 and drain the water from the water purification system 102. In some embodiments, the controller 104 (FIG. 1) can be configured to change a state of the valve 172 (FIG. 2) instead of, or in addition to, the valve 212 to prevent water from depleting the filters in the pretreatment system 150 if a suspected problem is identified with the electro-deionization module 202.

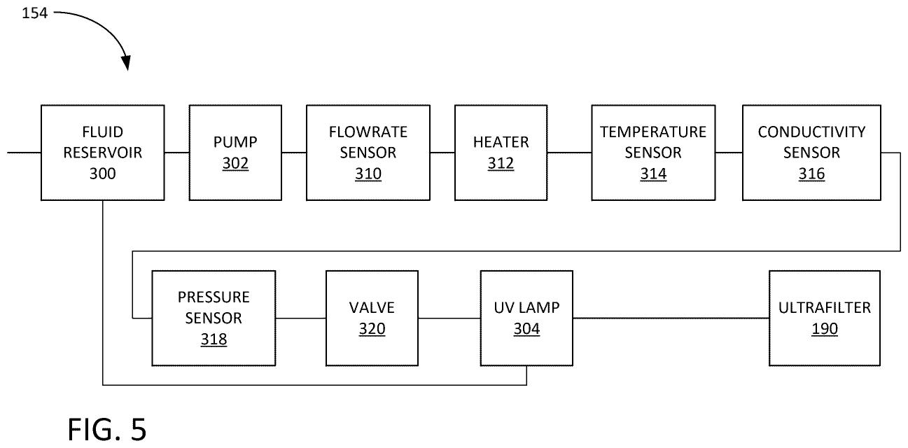

FIG. 5 is a schematic diagram of the distribution system 154 for the water purification system 102, according to some embodiments. In some embodiments, the distribution loop can include a fluid reservoir 300 and a pump 302 that is configured to circulate the water within the distribution system 154 to prevent stagnation. In some embodiments, the distribution system 154 includes a UV lamp 304. In some embodiments, the UV lamp 304 can kill bacteria, thereby facilitating prevention of bacterial formation. In some embodiments, the ultrafilter 190 is configured to be located downstream of the distribution system 154 and upstream of the cycler 106 (FIG. 1) to remove endotoxins produced by the UV lamp 304. In some embodiments, one or more ultrafilter 190 is configured to be located downstream of the distribution system 154 and upstream of the cycler 106 (FIG. 1) to remove endotoxins produced by the UV lamp 304. In some embodiments, two of the ultrafilter 190 (FIG. 2) are configured to be located downstream of the distribution system 154 and upstream of the cycler 106 (FIG. 1) to remove endotoxins produced by the UV lamp 304.

In some embodiments, the distribution system 154 includes the pump 302, a flowrate sensor 310, a heater 312, temperature sensor 314, a conductivity sensor 316, a pressure sensor 318, and a valve 320. In some embodiments, the valve 320 can be controlled to enable the water to either recirculate to the fluid reservoir 300 or to be provided to the ultrafilter 190. In some embodiments, the valve 192 (FIG. 2) is disposed downstream of the ultrafilter 190 to drain water if necessary. In some embodiments, the valve 194 (FIG. 2) is also disposed downstream of the ultrafilter 190 (FIG. 2) to control whether purified water is provided from the outlet 166 (FIG. 2) to the cycler 106 (FIG. 1).

With reference to FIGS. 3-5 collectively, in some embodiments, the water purification system 102 is configured to control ion removal from the source water. In some embodiments, the conductivity sensors (conductivity sensor 170, conductivity sensor 208, conductivity sensor 214, and conductivity sensor 316) can be used to assess whether the filtration steps in pretreatment system 150, treatment system 152, and distribution system 154 are working properly. At conductivity sensor 170, the conductivity of the source water is determined. At conductivity sensor 208, the conductivity of the water downstream of the reverse osmosis membrane 200 is determined. At conductivity sensor 214, the conductivity of the water downstream of the electro-deionization module 202 is determined. At conductivity sensor 316, the conductivity of the water in the distribution loop is determined. At each of these locations, the conductivity of the water should be trending downward. If at any of the locations downstream of the conductivity sensor 170 the conductivity is not decreasing, this can indicate a problem in the system and that ions are not being properly removed from the water. In some embodiments, the controller 104 can control one or more of the valve 172, the valve 188, the valve 192, the valve 194, the valve 212, the valve 216, or the valve 320 to drain the water from the system. As discussed above, the location of the valve being opened will be selected by the controller 104 to prevent unnecessary usage of the components of the water purification system 102 when an error condition has been identified.

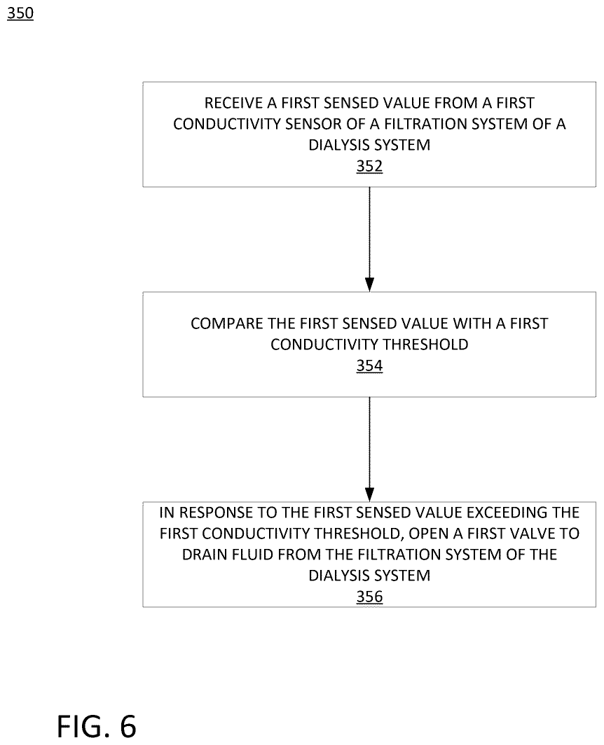

FIG. 6 is a flowchart of a method 350 for controlling a dialysis system (e.g., the dialysis system 100 of FIG. 1), according to some embodiments.

At block 352, the method 350 includes receiving, by a controller for a filtration system of a dialysis system (e.g., the controller 104 of the water purification system 102 (FIG. 1)), a first sensed value for a fluid from a first conductivity sensor of the filtration system. In some embodiments, the first conductivity sensor can be one of the conductivity sensor 170 (FIG. 2), the conductivity sensor 208 (FIG. 4), the conductivity sensor 214 (FIG. 4), or the conductivity sensor 316 (FIG. 5).

At block 354, the method 350 includes comparing, by the controller 104, the first sensed value with a first conductivity threshold.

At block 356, the method 350 includes, in response to the first sensed value being greater than the first conductivity threshold, opening the first valve to drain the fluid from the filtration system. In some embodiments, the first valve can be one of the valve 172 (FIG. 2), the valve 188 (FIG. 2), the valve 192 (FIG. 2), the valve 194 (FIG. 2), the valve 212 (FIG. 4), the valve 216 (FIG. 4), or the valve 320 (FIG. 5).

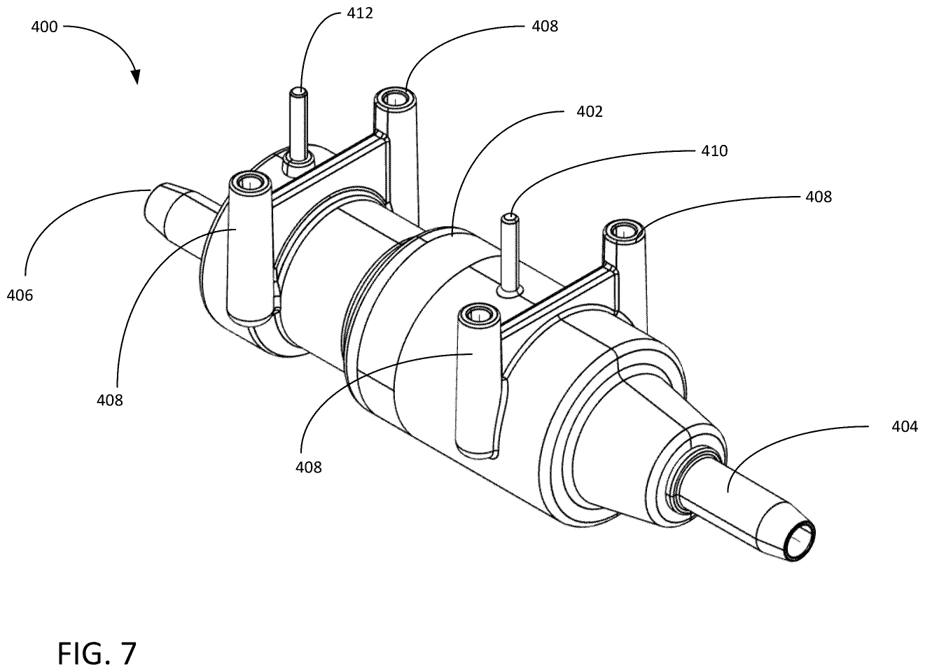

FIG. 7 shows a perspective view of a conductivity sensor 400, according to some embodiments. The conductivity sensor 400 can be used as one of the conductivity sensors shown and described above in a dialysis system 100. For example, the conductivity sensor 400 can be used in place of at least one of the conductivity sensor 170 (FIG. 2), the conductivity sensor 208 (FIG. 4), the conductivity sensor 214 (FIG. 4), or the conductivity sensor 316 (FIG. 5).

The conductivity sensor 400 includes a housing 402. A fluid inlet 404 is disposed at a first end of the housing 402. A fluid outlet 406 is disposed at a second end of the housing 402, the second end being opposite the first end. The fluid inlet 404 is fluidly connected to the fluid outlet 406. In some embodiments, the fluid inlet 404 is configured to be connected to a conduit in the water purification system 102. In some embodiments, the fluid outlet 406 is also configured to be connected to a conduit in the water purification system 102. In some embodiments, the fluid inlet 404 and associated conduit is configured to be upstream of the fluid outlet 406 and associated conduit. In some embodiments, a flow path of the fluid through the conductivity sensor 400 is from the fluid inlet 404 to the fluid outlet 406.

A plurality of output connectors 408 also extend from the housing 402. As a result, the conductivity sensor 400 can be connected to a processor via the plurality of output connectors 408. That is, in some embodiments, the processor can be secured to the plurality of output connectors 408. An electrode connection 410 extends from the housing 402. An electrode 412 also extends from the housing 402.

In some embodiments, the conductivity sensor 400 can be designed to measure conductivities within a conductivity sensing range of 0.5 to 150 μS/cm (micro Siemens per centimeter). In some embodiments, the conductivity sensor 400 can be designed to have an accuracy of +/−0.1 μS/cm.

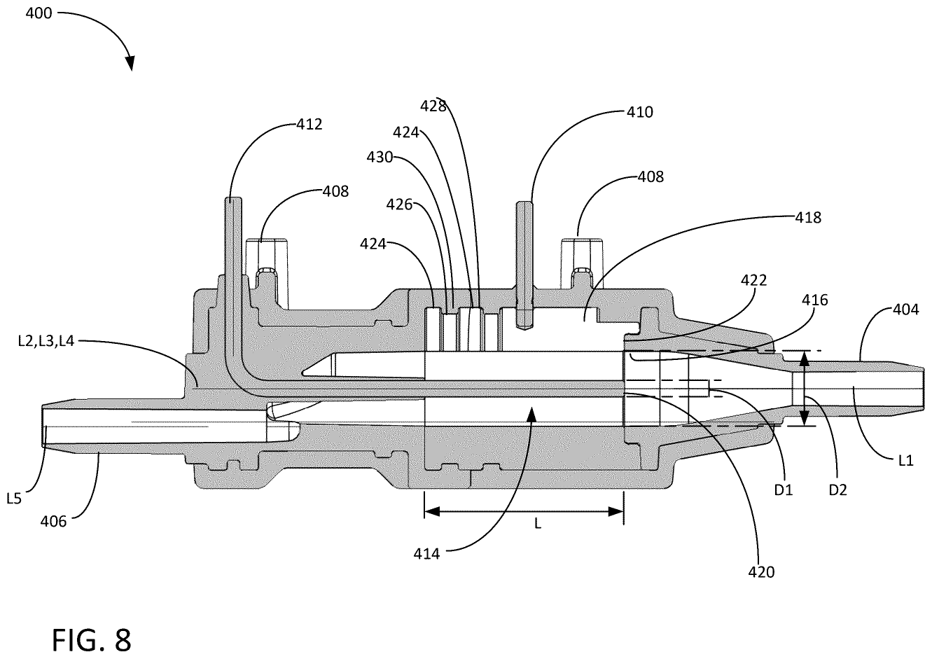

FIG. 8 shows a sectional view of the conductivity sensor 400 of FIG. 7, according to some embodiments.

In some embodiments, the housing 402 includes a chamber 414 disposed between the fluid inlet 404 and the fluid outlet 406. The chamber 414 is fluidly connected to the fluid inlet 404 and the fluid outlet 406. The chamber 414 includes an inner wall 416. At least a portion of the inner wall 416 of the chamber 414 is formed by an inner surface of an electrode 418. In some embodiments, electrode 412 extends at least partially through the chamber 414 and through an interior of the electrode 418. In some embodiments, an end 420 of the electrode 412 extends to be adjacent to an end 422 of the electrode 418 within the housing 402. In some embodiments, the end 420 and the end 422 can be flush with each other.

In some embodiments, electrode 412 can be a bent rod, an end of the electrode 412 including the bend including an electrode connection. In some embodiments, the bent end of the electrode 412 can extend to be adjacent to the end 420 of electrode 412. In other embodiments, electrode 412 can be a straight rod, the electrode 412 having a separate electrode connection in contact with the electrode 412.

In some embodiments, a longitudinal axis L1 of the fluid inlet 404 is coaxial with a longitudinal axis L2 of the chamber 414. In some embodiments, a longitudinal axis L3 of the electrode 412 and a longitudinal axis L4 of the electrode 418 are coaxial. In some embodiments, the longitudinal axes L1, L2, L3, and L4 are coaxial.

In some embodiments, a longitudinal axis L5 of the fluid outlet 406 is offset from the longitudinal axes L1, L2, L3, and L4. In some embodiments, the longitudinal axis L5 is parallel to the longitudinal axes L1, L2, L3, and L4.

In some embodiments, the electrode 412 is a solid piece of material. In some embodiments, the electrode 412 is a wire. In some embodiments, the electrode 412 has an outer diameter D1. In some embodiments, the electrode 412 is a rod. In other embodiments, the electrode 412 is a pin. In some embodiments, the electrode 412 is a cylindrically shaped object. That is, an outer surface of the electrode 412 can be cylindrically shaped.

In some embodiments, the electrode 418 is electrically connected to the electrode connection 410. In some embodiments, the electrode 418 can have a cylindrical interior. In some embodiments, the arrangement of the electrode 412 and the electrode 418 can thus be described as being coaxial cylinders. In some embodiments, the electrode 418 has an inner diameter D2.

In some embodiments, the outer diameter D1 of the electrode 412 is smaller than the inner diameter D2 of the electrode 418. That is, in some embodiments, the electrode 412 is spaced from the electrode 418 to prevent direct contact between the electrode 412 and the electrode 418. In some embodiments, the electrode 412 is additionally spaced from the electrode 418 to let the fluid to flow in the space in chamber 414 not occupied by electrode 412 and electrode 418.

In some embodiments, the electrode 418 can have an outer surface including a series of protrusions 424. A series of grooves 426 is defined between the protrusions 424. In some embodiments, the protrusions 424 and grooves 426 can be configured to mate with an offset series of grooves 428 and protrusions 430 formed in the housing 402 when the electrode 418 is installed within the housing 402. In some embodiments, this mating engagement can ensure that the electrode 418 is fixed relative to the housing 402.

In some embodiments, a portion of the electrode 412 extends through an interior of the electrode 418. In some embodiments, a length of the current path can be defined as L. In some embodiments, the length L is based on the distance through which the electrode 412 extends inside of the electrode 418. In some embodiments, the length of the current path L can be given by the distance between the electrodes, e.g., electrode 412 and electrode 418. In this regard, the length of the current path L can be related to the area of the cell, and to the area that can be intercepted by the electrical current, affecting the total responsivity of the conductivity sensor 400.

In some embodiments, the electrode 412 can be made of a conductive metal. In some embodiments, the conductive metal can include titanium.

In some embodiments, the electrode 418 can be made of a conductive metal. In some embodiments, the conductive metal can include titanium.

In some embodiments, the housing 402 can be made of an electrically insulative material.

In some embodiments, a cell constant for the conductivity sensor 400 can be determined based on a geometry of the electrode 412 and the electrode 418. For example, the cell constant can be determined based on the outer diameter D1 of the electrode 412 and the inner diameter D2 of the electrode 418. In some embodiments, the cell constant can also be determined based on the length L of the current path. In some embodiments, the cell constant (K) can be determined using the following equation:

$$K = \frac{ln ( \frac{D 2}{D 1} )}{2 π L}$$

### EXAMPLE

Several probes were tested along with two reference probes to illustrate effectiveness of the probes according to the present disclosure in water at an ultralow conductivity condition having a conductivity of 1 μS/cm, a temperature of 10° C., and a flowrate of 50 mL/min. The results are shown in Table 1.

TABLE 1
Measured Conductivity, Temperature, and
Flowrate for 1 μS/cm, 10° C., 50 mL/min

| | | Con- | | Flow |
|---|---|---|---|---|
| | | ductivity | Temperature | Rate |
| Run | Probe | [μS/cm] | [° C.] | [mL/min] |
| 1 | Reference 1 | 0.789456 | 9.8862 | 37.1448 |
| 1 | Probe 1 | 0.905334 | 9.9094 | 37.1448 |
| 1 | Probe 2 | 0.894016 | 9.9519 | 37.1448 |
| 1 | Probe 3 | 0.899230 | 10.1158 | 37.1448 |
| 1 | Probe 4 | 0.905917 | 9.9740 | 37.1448 |
| 1 | Probe 5 | 0.896917 | 9.8611 | 37.1448 |
| 1 | Probe 6 | 0.898568 | 9.9459 | 37.1448 |
| 1 | Reference 2 | 0.809464 | 9.3641 | 37.1448 |
| 2 | Reference 1 | 0.777200 | 9.9779 | 49.6588 |
| 2 | Probe 1 | 0.880471 | 10.0117 | 49.6588 |
| 2 | Probe 2 | 0.871514 | 10.0566 | 49.6588 |
| 2 | Probe 3 | 0.875614 | 10.2170 | 49.6588 |
| 2 | Probe 4 | 0.882634 | 10.0674 | 49.6588 |
| 2 | Probe 5 | 0.873456 | 9.9449 | 49.6588 |
| 2 | Probe 6 | 0.875000 | 10.0209 | 49.6588 |
| 2 | Reference 2 | 0.787458 | 9.4775 | 49.6588 |
| 3 | Reference 1 | 0.847417 | 9.9167 | 52.6288 |
| 3 | Probe 1 | 0.837446 | 9.9657 | 52.6288 |
| 3 | Probe 2 | 0.840326 | 10.0938 | 52.6288 |
| 3 | Probe 3 | 0.846136 | 9.9336 | 52.6288 |
| 3 | Probe 4 | 0.838201 | 9.7878 | 52.6288 |
| 3 | Probe 5 | 0.836757 | 9.8695 | 52.6288 |
| 3 | Probe 6 | 0.754742 | 9.3139 | 52.6288 |
| 3 | Reference 2 | 0.847417 | 9.9167 | 52.6288 |

The tested probes all showed to successfully emulate the reference conductivity probes, establishing that the determined conductivities had a minimum resolution of 0.1 μS/cm and a temperature accuracy within +/−2° C.

The probes were tested along with the two reference probes to illustrate effectiveness of the probes according to the present disclosure in water at an ultralow conductivity condition having a conductivity of 5.5 μS/cm, a temperature of 25° C., and a flowrate of 325 mL/min. The results are shown in Table 2.

TABLE 2
Measured Conductivity, Temperature, and Flowrate
for 5.5 μS/cm, 25° C., 325 mL/min

| | | | Temper- | |
|---|---|---|---|---|
| | | Conductivity | ature | Flow Rate |
| Run | Probe | [μS/cm] | [° C.] | [mL/min] |
| 1 | Reference 1 | 5.48342 | 24.9151 | 319.295 |
| 1 | Probe 1 | 6.02038 | 25.1202 | 319.295 |
| 1 | Probe 2 | 5.94901 | 25.1787 | 319.295 |
| 1 | Probe 3 | 6.00248 | 25.4283 | 319.295 |
| 1 | Probe 4 | 6.04939 | 25.3060 | 319.295 |
| 1 | Probe 5 | 5.99639 | 25.2341 | 319.295 |
| 1 | Probe 6 | 5.99879 | 25.3210 | 319.295 |
| 1 | Reference 2 | 5.55497 | 24.5968 | 319.295 |
| 2 | Reference 1 | 5.42620 | 25.0485 | 313.378 |
| 2 | Probe 1 | 5.94467 | 25.2686 | 313.378 |
| 2 | Probe 2 | 5.87512 | 25.3214 | 313.378 |
| 2 | Probe 3 | 5.92503 | 25.5703 | 313.378 |
| 2 | Probe 4 | 5.97302 | 25.4482 | 313.378 |
| 2 | Probe 5 | 5.92159 | 25.3788 | 313.378 |
| 2 | Probe 6 | 5.92602 | 25.4628 | 313.378 |
| 2 | Reference 2 | 5.50028 | 24.7510 | 313.378 |
| 3 | Reference 1 | 5.44764 | 25.0959 | 329.629 |
| 3 | Probe 1 | 5.96359 | 25.3453 | 329.629 |
| 3 | Probe 2 | 5.89702 | 25.4003 | 329.629 |
| 3 | Probe 3 | 5.94929 | 25.6448 | 329.629 |
| 3 | Probe 4 | 5.99645 | 25.5162 | 329.629 |
| 3 | Probe 5 | 5.94547 | 25.4380 | 329.629 |
| 3 | Probe 6 | 5.94517 | 25.5204 | 329.629 |
| 3 | Reference 2 | 5.53083 | 24.8639 | 329.629 |
| 3 | Reference 1 | 5.50053 | 24.8202 | 334.092 |

The tested probes all showed to successfully emulate the reference conductivity probes, establishing that the determined conductivities had a minimum resolution of 0.1 μS/cm and a temperature accuracy within +/−2° C.

The probes were tested along with the two reference probes to illustrate effectiveness of the probes according to the present disclosure in water at an ultralow conductivity condition having a conductivity of 10 μS/cm, a temperature of 10° C., and a flowrate of 50 mL/min. The results are shown in Table 3.

TABLE 3
Measured Conductivity, Temperature, and Flowrate
for 10 μS/cm, 10° C., 50 mL/min

| | | | Temper- | |
|---|---|---|---|---|
| | | Conductivity | ature | Flow Rate |
| Run | Probe | [μS/cm] | [° C.] | [mL/min] |
| 1 | Reference 1 | 6.92194 | 9.9334 | 22.2488 |
| 1 | Probe 1 | 7.70136 | 10.0228 | 22.2488 |
| 1 | Probe 2 | 7.63428 | 10.0721 | 22.2488 |
| 1 | Probe 3 | 7.66079 | 10.2402 | 22.2488 |
| 1 | Probe 4 | 7.72357 | 10.0953 | 22.2488 |
| 1 | Probe 5 | 7.65289 | 10.0114 | 22.2488 |
| 1 | Probe 6 | 7.65570 | 10.0849 | 22.2488 |
| 1 | Reference 2 | 7.00803 | 9.5320 | 22.2488 |
| 2 | Reference 1 | 6.99992 | 9.9778 | 41.5925 |
| 2 | Probe 1 | 7.79492 | 10.0392 | 41.5925 |
| 2 | Probe 2 | 7.72081 | 10.0987 | 41.5925 |
| 2 | Probe 3 | 7.74603 | 10.2394 | 41.5925 |
| 2 | Probe 4 | 7.80758 | 10.0976 | 41.5925 |
| 2 | Probe 5 | 7.73599 | 9.9923 | 41.5925 |
| 2 | Probe 6 | 7.73782 | 10.0638 | 41.5925 |
| 2 | Reference 2 | 7.06675 | 9.5020 | 41.5925 |
| 3 | Reference 1 | 7.02761 | 10.0997 | 48.5916 |
| 3 | Probe 1 | 7.80906 | 10.1737 | 48.5916 |
| 3 | Probe 2 | 7.75118 | 10.2366 | 48.5916 |
| 3 | Probe 3 | 7.78150 | 10.4015 | 48.5916 |
| 3 | Probe 4 | 7.84655 | 10.2475 | 48.5916 |
| 3 | Probe 5 | 7.78026 | 10.1564 | 48.5916 |
| 3 | Probe 6 | 7.76585 | 10.2325 | 48.5916 |
| 3 | Reference 2 | 6.97497 | 9.6474 | 48.5916 |

The tested probes all showed to successfully emulate the reference conductivity probes, establishing that the determined conductivities had a minimum resolution of 0.1 μS/cm and a temperature accuracy within +/−2° C.

The terminology used herein is intended to describe embodiments and is not intended to be limiting. The terms “a,” “an,” and “the” include the plural forms as well, unless clearly indicated otherwise. The terms “comprises” and/or “comprising,” when used in this Specification, specify the presence of the stated features, integers, steps, operations, elements, and/or components, but do not preclude the presence or addition of one or more other features, integers, steps, operations, elements, and/or components.

It is to be understood that changes may be made in detail, especially in matters of the construction materials employed and the shape, size, and arrangement of parts without departing from the scope of the present disclosure. This Specification and the embodiments described are examples, with the true scope and spirit of the disclosure being indicated by the claims that follow.

1. A conductivity sensor comprising:
a housing comprising:
a fluid inlet;
a fluid outlet; and
a chamber having an inner wall;
wherein the chamber is located between the fluid inlet and the fluid outlet;
a first electrode;
wherein at least a portion of the first electrode is located in the chamber;
wherein the first electrode has a first longitudinal axis;
wherein the first electrode does not form any portion of the inner wall of the chamber; and
a second electrode;
wherein the second electrode forms at least a portion of the inner wall of the chamber;
wherein the second electrode has a second longitudinal axis;
wherein the first longitudinal axis is coaxial with the second longitudinal axis;
wherein a flow path of a fluid through the conductivity sensor is from the fluid inlet through the chamber to the fluid outlet.

2. The conductivity sensor of claim 1, wherein the fluid inlet has a third longitudinal axis;
wherein the fluid outlet has a fourth longitudinal axis;
wherein the third longitudinal axis is parallel to the fourth longitudinal axis; and
wherein the third longitudinal axis is offset from the fourth longitudinal axis.

3. The conductivity sensor of claim 2, wherein the third longitudinal axis is coaxial with the first longitudinal axis.

4. The conductivity sensor of claim 1, wherein an outer surface of the second electrode comprises a plurality of protrusions;
wherein an inner surface of the housing comprises a plurality of grooves;
wherein the plurality of grooves is configured to receive the plurality of protrusions when the second electrode is installed in the housing.

5. The conductivity sensor of claim 1, wherein at least one of the first electrode or the second electrode includes titanium.

6. The conductivity sensor of claim 1, wherein the housing comprises an output connector configured to be connected to a processor.

7. The conductivity sensor of claim 6, wherein the processor is configured to be secured to the housing.

8. The conductivity sensor of claim 1, wherein a first end of the first electrode is configured to be disposed adjacent to a first end of the second electrode;
wherein the first end of the second electrode is disposed downstream of a second end of the second electrode.

9. The conductivity sensor of claim 1, wherein the first electrode is a first cylindrically shaped electrode having a first diameter,
wherein the second electrode is a second cylindrically shaped electrode having a second diameter, and
wherein the second diameter is greater than the first diameter.

10. The conductivity sensor of claim 1, wherein the conductivity sensor is configured to bring the fluid flowing through the chamber into contact with the first electrode and the second electrode.

11. A dialysis system comprising:
a water purification system configured to purify a fluid for use in dialysis;
wherein the water purification system comprises:
a conductivity sensor configured to sense a conductivity of the fluid for use in dialysis,
wherein the conductivity sensor comprises:
a housing comprising:
a fluid inlet;
a fluid outlet; and
a chamber having an inner wall;
wherein the chamber is located between the fluid inlet and the fluid outlet;
a first electrode;
wherein at least a portion of the first electrode is located in the chamber,
wherein the first electrode does not form any portion of the inner wall of the chamber; and
a second electrode;
wherein the second electrode forms at least a portion of the inner wall of the chamber;
wherein a flow path of the fluid for use in dialysis through the conductivity sensor is from the fluid inlet through the chamber to the fluid outlet.

12. The dialysis system of claim 11, wherein the first electrode has a first longitudinal axis;
wherein the second electrode has a second longitudinal axis;
wherein the first longitudinal axis is coaxial with the second longitudinal axis; and
wherein the fluid outlet has a third longitudinal axis;
wherein the third longitudinal axis is coaxial with the first longitudinal axis.

13. The dialysis system of claim 12, wherein the fluid inlet has a fourth longitudinal axis; and wherein the third longitudinal axis is offset from the fourth longitudinal axis.

14. The dialysis system of claim 11, wherein an outer surface of the second electrode comprises a plurality of protrusions;
wherein an inner surface of the housing comprises a plurality of grooves;
wherein the plurality of grooves is configured to receive the plurality of protrusions when the second electrode is installed in the housing.

15. The dialysis system of claim 11, wherein at least one of the first electrode or the second electrode includes titanium.

16. The dialysis system of claim 11, wherein the housing comprises an output connector configured to be connected to a processor configured to secure the processor to the housing.

17. The dialysis system of claim 16, wherein the processor is configured to be secured to the housing.

18. The dialysis system of claim 11, wherein a first end of the first electrode is configured to be disposed adjacent a first end of the second electrode, wherein the first end of the second electrode is disposed downstream of a second end of the second electrode.

19. The dialysis system of claim 11, comprising a second conductivity sensor configured to sense the conductivity of the fluid for use in dialysis at a different location in the water purification system than the conductivity sensor.

20. The dialysis system of claim 11, wherein the conductivity sensor is configured to determine a first error condition in which a concentration of ions in the fluid for use in dialysis is greater than an ion concentration threshold.
